(19) United States
(12) Patent Application Publication
(10) Pub. No.: US 2008/0312448 A1
(43) Pub. Date: Dec. 18, 2008

(54) Process for Synthesizing Remifentanil

(76) Inventor: Brian K. Cheng, Chesterfield, MO (US)

Correspondence Address: Mallinckrodt Inc., 675 McDonnell Boulevard, HAZELWOOD, MO, 63042, US

(21) Appl. No.: 12/161,171

(22) PCT Filed: Jan. 8, 2007
(86) PCT No.: PCT/US07/00526

§ 371 Date: Jul. 17, 2008

Related U.S. Application Data
(60) Provisional application No. 60/761,480, filed on Jan. 24, 2006.

(51) Int. Cl. C07D 211/06

(52) U.S. Cl. 546/244

(57) ABSTRACT

A process for synthesizing remifentanil or carfentanil, as well as intermediates for use in the preparation of synthetic opiate or opioid compounds. The process comprising reacting a 4-amino 4-carbamyl piperidine with a base in a closed reaction chamber at elevated temperature and pressure to form to intermediate which may be esterified with an alcohol, alkylated, and acylated to produce a synthetic opiate or opioid compound.

### FIELD OF THE INVENTION

The present invention generally relates to a process for synthesizing opiate or opioid analgesics and anesthetics, and precursors thereof. In particular, the present invention relates to processes of synthesizing intermediates for use in the preparation of synthetic opiate or opioid compounds such as, for example, remifentanil, carfentanil, sulfentanil, fentanyl, and alfentanil. In particular, the present invention relates to a preparation process with fewer steps, reduced costs, improved safety, and higher efficiency than processes known in the art for producing remifentanil and carfentanil.

### BACKGROUND OF THE INVENTION

Analgesics and anesthetics, such as remifentanil and carfentanil, have been prepared in synthetic processes comprising six and seven steps. Examples of such processes are outlined in U.S. Pat. Nos. 5,106,983 and 5,019,583. However, these syntheses often require multiple protection and deprotection steps of reactive moieties, resulting in increased process costs due to reduced production efficiency and additional material costs. These processes also use cyanide compounds which substantially increase safety and environmental concerns, waste disposal cost, and require EPA registration.

A process without the use of cyanide compounds would improve safety, reduce cost, and eliminate the need for EPA registration. A process with fewer process steps would be beneficial in improving process efficiencies, and reducing the cost of synthesizing analgesics and anesthetics.

### SUMMARY OF THE INVENTION

Among the several features of the present invention, therefore, can be noted the provision of a process for synthesizing intermediates for use in the preparation of synthetic opiate or opioid compounds such as, for example, remifentanil, carfentanil, sulfentanil, fentanyl, and alfentanil; the provision of preparing an analgesic or anesthetic; the provision of a process that requires fewer steps for synthesizing remifentanil; the provision of a process that requires fewer steps for synthesizing carfentanil; and the provision of such a process wherein remifentanil is prepared from a 4-amino 4-carbamyl piperidine.

Briefly, therefore, the present invention is directed to a process for the preparation of an analgesic or anesthetic. A compound (I) having the formula:

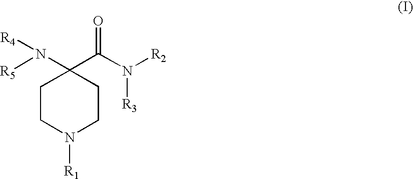

is reacted with a base in the presence of a solvent to form intermediate compound (II):

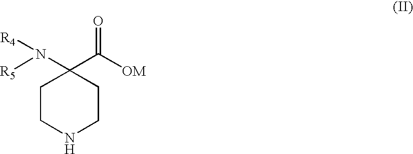

wherein R₁, R₂, R₃, R₄, and R₅ are independently selected from the group consisting of hydrogen, hydrocarbyl, and substituted hydrocarbyl and M is hydrogen or a cation. The intermediate compound (II) is reacted with alcohol, R₆OH, to form an intermediate compound (III):

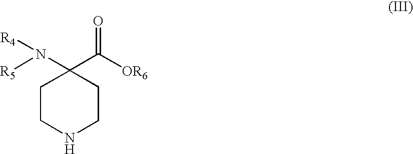

wherein R₆ is a hydrocarbyl or substituted hydrocarbyl. The intermediate compound (III) is reacted with an alkylating agent to form an intermediate compound (IV):

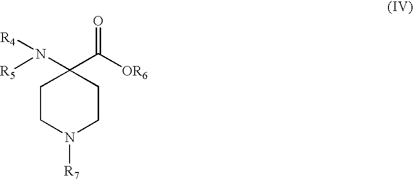

wherein R₇ is a hydrocarbyl or substituted hydrocarbyl. The intermediate compound (IV) is reacted with an acylating agent to form an analgesic or anesthetic compound (V) having the formula:

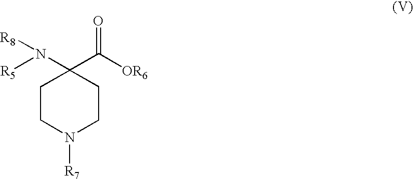

wherein R₈ is —C(O)—R₉, and R₉ is hydrocarbyl or substituted hydrocarbyl.

In another aspect, the invention is directed to a process for synthesizing an intermediate useful in the synthesis of opiate or opioid analgesics or anesthetics. The process comprises reacting compound (I) having the formula:

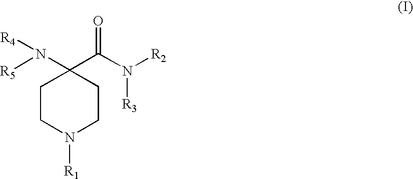

with a base in the presence of a solvent to form intermediate compound (II) having the formula:

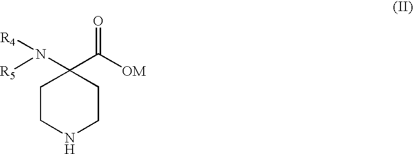

wherein R₁, R₂, R₃, R₄, and R₅ are independently selected from the group consisting of hydrogen, hydrocarbyl, and substituted hydrocarbyl and M is hydrogen or a cation. Compound (II) is a useful intermediate in the production of opiate or opioid analgesics or anesthetics.

In another aspect, the invention is directed to a process for synthesizing intermediates of opiate or opioid analgesics or anesthetics. The process comprises reacting the intermediate compound (II) with alcohol, R₆OH, to form an intermediate compound (III):

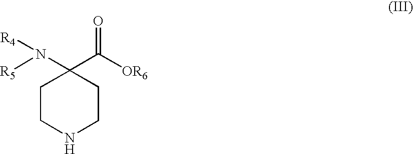

wherein R₄, and R₅ are independently selected from the group consisting of hydrogen, hydrocarbyl, and substituted hydrocarbyl; and R₆ is selected from the group consisting of hydrocarbyl and substituted hydrocarbyl. Reacting the intermediate compound (III) with an alkylating agent to form an intermediate compound (IV):

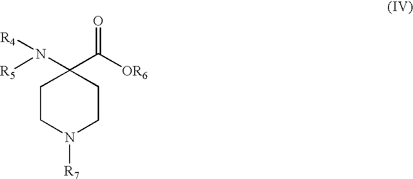

wherein R₇ is a hydrocarbyl or substituted hydrocarbyl. The intermediate compound (IV) is reacted with an acylating agent to form compound (V) having the formula:

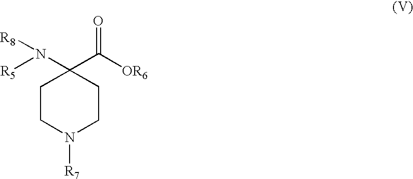

wherein R₈ is —C(O)—R₉, and R₉ is hydrocarbyl or substituted hydrocarbyl. Each intermediate may be used in the preparation of synthetic opiate or opioid compounds.

Other aspects and features of this invention will be in part apparent and in part pointed out hereinafter.

### DETAILED DESCRIPTION

In accordance with the present invention, an improved process for synthesizing analgesics or anesthetics has been discovered. The improved process reduces the number of process steps required to synthesize the analgesics or anesthetics and avoids the use of cyanide compounds. The process also improves yield of synthesized analgesic or anesthetic product as compared to processes known in the art.

In one embodiment, the process of the present invention results in the synthesis of a compound having the formula (V):

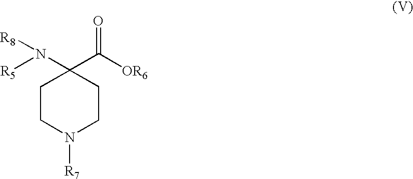

wherein R₈ is —C(O)R₉, R₅ is hydrogen, hydrocarbyl or substituted hydrocarbyl, and R₆, R₇, and R₉ are independently selected from hydrocarbyl or substituted hydrocarbyl.

In another embodiment, R₇ is hydrocarbyl or substituted hydrocarbyl, R₅ is a phenyl or substituted phenyl, R₈ is a carbonyl alkyl, and R₆ is hydrocarbyl or substituted hydrocarbyl.

In one embodiment, the present invention can be used to synthesize remifentanil, chemically identified as 3-[4-methoxycarbonyl-4-[(1-oxopropyl)phenylamino]-1-piperidine]propanoic acid methyl ester, having the formula (VI), utilizing a 4-amino 4-carbamyl piperidine starting material.

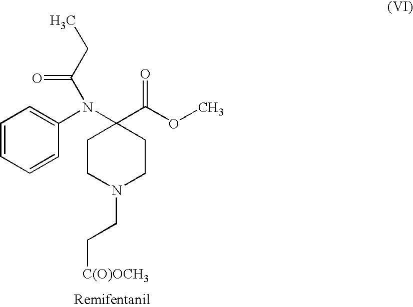

In another embodiment, the present invention can be used to synthesize carfentanil, chemically identified as 4((1-oxopropyl)phenylamino)-1-(2-phenylethyl)-4-piperidinecarboxylic acid, methyl ester, having the formula (VII), by utilizing a 4-amino 4-carbamyl piperidine starting material.

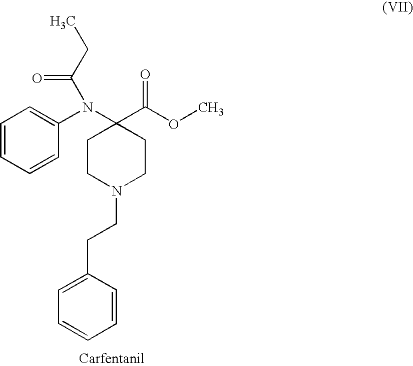

The improved process of the present invention for synthesizing opiate or opioid analgesics and anesthetics includes the synthesis of a series of intermediates, each of which may be used in the preparation of synthetic opiate or opioid compounds. Scheme 1, below, illustrates a first step in the process wherein a 4-amino 4-carbamyl piperidine, compound (I), is hydrolyzed to form intermediate compound (II).

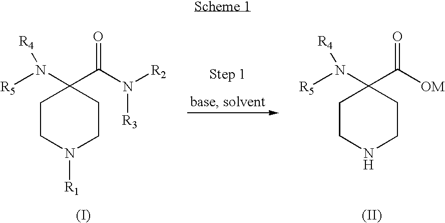

In one embodiment, compound (I) is mixed with a base in the presence of a solvent to form intermediate compound (II), wherein R₁, R₂, R₃, R₄, and R₅ are independently selected from the group consisting of hydrogen, hydrocarbyl, and substituted hydrocarbyl and M is hydrogen or a cation.

Preferably, R₁ is selected from the group consisting of H, R₁₀O(O)C—, R₁₁O(O)C—, R₁₁R₁₀—, R₁₀O(O)CR₁₂—, R₁₁R₁₀O(O)CR₁₂—, R₁₁(O)COR₁₂—, R₁₀(O)COR₁₂—, R₁₁R₁₀(O)COR₁₂—, R₁₀(O)CR₁₃OR₁₂—, and R₁₁(O)CR₁₃OR₁₂—, wherein R₁₀, R₁₂, and R₁₃ are independently hydrocarbyl or substituted hydrocarbyl, and R₁₁ is heterocyclic. Preferably, R₁₀, R₁₂, and R₁₃ are independently substituted or non-substituted alkyl, alkoxy, alkenyl, alkenyloxy, and aryl groups, R₁₁ is a 5 to 7 member heterocyclic comprising 1 to 5 hetero-atoms selected from oxygen, sulfur, and nitrogen; more preferably, R₁₀, R₁₂, and R₁₃ are independently linear or branched alkyl, alkoxy, alkenyl, alkynyl and alkenyloxy groups having about 1 to about 18 carbon atoms, and R₁₁ is a 5- to 7-member cycloalkyl; still more preferably, R₁ is H, R₁₀(O)COR₁₂— or R₁₀O(O)C—.

Preferably, R₂, R₃, R₄, and R₅ are independently selected from the group consisting of H, cycloalkyl, substituted cycloalkyl, heterocyclic, R₁₄OR₁₅—, and R₁₆R₁₅—, wherein R₁₄ and R₁₅ are independently hydrocarbyl or substituted hydrocarbyl, and R₁₆ is selected from the group consisting of cycloalkyl, substituted cycloalkyl, and heterocyclic. Preferably, R₁₄ and R₁₅ are independently substituted and non-substituted alkyl, alkoxy, alkenyl, alkenyloxy, and aryl groups, R₁₆ is a cycloalkyl comprising 3 to 6 carbon atoms, substituted cycloalkyl comprising 3 to 6 carbon atoms, or 5 to 7 member heterocyclic comprising 1 to 5 hetero-atoms selected from oxygen, sulfur, and nitrogen; more preferably, R₁₄ and R₁₅ are independently H, substituted and non-substituted alkyl, alkoxy, and aryl groups; still more preferably, R₂, R₃, R₄, and R₅ are independently selected from H, lower-alkyl, and phenyl.

M corresponds to the resulting cation of the base or cations resulting from the combination of base used in the reaction mixture. Preferably, M is an alkali or alkaline earth metal cation, more preferably, M is a sodium, potassium, or lithium cation.

In one embodiment, the reaction occurs at elevated temperature. In another embodiment, the temperature of the reaction mixture during the reaction ranges from about 120° C. to about 200° C. Preferably, the temperature ranges from about 145° C. to about 175° C. In one embodiment the reaction mixture is permitted to react from about 4 hours to about 48 hours. Preferably, reaction time is from about 12 hours to about 24 hours.

In one embodiment, the reaction occurs in a closed reaction chamber at elevated temperature and pressure. In another embodiment the reaction is carried out in a closed reaction vessel able to withstand elevated temperature and pressure, for example a Parr stirred pressure reactor. The increased temperature in the closed reaction vessel results in an elevated pressure within the vessel. In one embodiment the pressure within the vessel during the reaction ranges from about 65 p.s.i. to about 165 p.s.i.

The solvent used in the reaction mixture can include water and/or one or more organic solvents. Examples of organic solvents include, but are not limited to, cyclic ethers; alkyl ethers; alkanes; aromatic hydrocarbons such as benzene, toluene, and xylene; alkanols, for example, methanol, ethanol, isopropanol, n-propanol, 1-butanol, tert-butanol, and the like ethers such as 1,4-dioxane, tetrahydrofuran (THF), 1,1-oxybisethane, and the like; and mixtures thereof. Preferably the solvent is an alkanol; more preferably the solvent is an alkanol with 1 to 3 carbon atoms. Still more preferably the solvent is ethanol, isopropanol, n-propanol, or methanol.

The reaction mixture contains a base to hydrolyze compound (I). The base may be any base capable of hydrolyzing the amide group of compound (I). Preferably the base is a strong base; more preferably the base is a metal hydroxide, metal hydride, amine, ammonium hydroxide, or quaternary alkyl ammonium hydroxide. Still more preferably the base is a metal hydroxide such as sodium hydroxide, potassium hydroxide or lithium hydroxide. The base may also comprise a mixture of different bases.

In one embodiment, the reaction mixture comprises about 1 molar equivalent to about 6 molar equivalents of base to 1 molar equivalent of compound (I). Preferably, the reaction mixture is charged with about 3 to about 5 molar equivalents of base to 1 molar equivalent of compound (I).

The solvent to reaction mixture ratio on a weight/volume basis is from about 1:2 to about 1:100, preferably, from about 1:1.5 to 1:5.

The reaction mixture may contain a catalyst such as a crown ether, quaternary ammonium salt, Lewis acid, or transition metal catalyst.

Optionally, acid may be added to compound (II) to produce a protic salt form of compound (II). In one embodiment the acid added is a strong acid. Preferably the acid is hydrochloric acid, sulfuric acid, nitric acid, phosphoric acid, hydroiodic acid, hydrobromic acid or a combination of such.

In one embodiment, compound (II) is isolated by cooling the reaction to ambient temperature, relieving the internal pressure of the vessel and thereafter, filtering, and drying the solid product.

In another embodiment, compound (II) is isolated by cooling the reaction to ambient temperature, relieving the internal pressure of the vessel, and thereafter, dissolving the solid product by acidification, precipitating the product by addition of base, and recovering compound (II) through filtration, washing, and drying.

Scheme 2, below, illustrates a second step in the process of the present invention wherein intermediate compound (III) is synthesized.

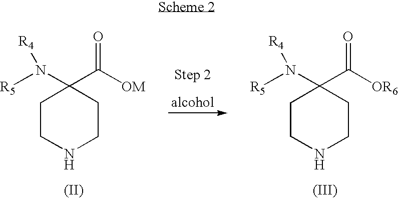

In Scheme 2, compound (II), is reacted with an alcohol, R₆OH to form intermediate compound (III), wherein R₆ is a hydrocarbyl or substituted hydrocarbyl.

Preferably, R₆ is selected from the group consisting of R₁₇OR₁₈—, R₁₉R₁₈—, or R₂₀R₁₈—, wherein R₁₇ and R₁₈ are independently hydrocarbyl or substituted hydrocarbyl groups, R₁₉ is aryl or substituted aryl, and R₂₀ is cycloalkyl, substituted cycloalkyl or heterocyclic. Preferably, R₁₇ and R₁₈ are independently substituted and non-substituted alkyl, alkenyl, and alkynyl groups wherein the hydrocarbon chain contains 1 to 18 carbon atoms, R₁₉ is aryl or substituted aryl, R₂₀ is cycloalkyl comprising 3 to 6 carbon atoms, substituted cycloalkyl comprising 3 to 6 carbon atoms, or 5 to 7 member heterocyclic comprising 1 to 5 hetero-atoms selected from oxygen, sulfur, and nitrogen; more preferably, R₁₇ and R₁₈ are independently substituted and non-substituted alkyl groups; still more preferably, R₆ is methyl, ethyl or propyl.

In one embodiment, the temperature of the reaction mixture during the reaction ranges from about 25° C. to about 80° C., preferably, from about 50° C. to about 70° C. The reaction mixture is permitted to react up to a few days. In one example, the reaction occurs from about 8 to about 100 hours, preferably, from about 24 hours to about 60 hours.

Desiccant may be used to enhance the rate of esterification of compound (II). Non-limiting examples of desiccant compounds include trimethyl orthoformate, sulfur trioxide, polyphosphoric acid, phosphorous pentoxide, molecular sieves, alumina, silica gel, sodium sulfate anhydrous, magnesium sulfate, and the like.

Catalyst may be used to enhance the reaction. The catalyst may be selected from the group commonly known as Bronsted acids. A Bronsted acid may be an inorganic acid, examples of which include, but are not limited to, sulfuric acid, hydrochloric acid, nitric acid, phosphoric acid, hydroiodic acid, hydrobromic acid, and hydrofluoric acid; or an organic acid, examples of which include, but are not limited to, methanesulfonic acid, toluenesulfonic acid, benzenesulfonic acid, trifluoroacetic acid, pentafluoroacetic acid, chloroacetic acid, dichloroacetic acid, trichloroacetic acid, and oxalic acid. The catalyst may also be selected from the group know as Lewis acids, examples of which include, but are not limited to, boron trifluoride, aluminum chloride, zinc chloride, tin chloride, titanium tetrachloride and solid acid like cationic resins, alumina, silica gel, and others known in the art.

In one embodiment, the reaction mixture comprises about 2 molar equivalents to about 100 molar equivalents of alcohol, optionally about 1 molar equivalent to about 5 molar equivalents of desiccant, and optionally about 1 molar equivalent to about 10 molar equivalents of catalyst per molar equivalent of compound (II).

In another embodiment, the reaction mixture is charged with about 4 molar equivalents to about 50 molar equivalents of alcohol, about 1 molar equivalent to about 3 molar equivalents of desiccant, and about 2 molar equivalents to about 4 molar equivalents of catalyst per molar equivalent of compound (II).

Depending on its physical properties, compound (III) may be purified and isolated by extraction, chromatography, distillation, or any combination of methods known in the art.

In one embodiment, compound (III) is isolated by the addition of base and water, followed by solvent extraction of compound (III) and finally drying by evaporation.

In another embodiment, compound (III) is isolated by cooling the reaction to below 10° C., adding triethylamine to precipitate the resulting anion of an appropriate Bronsted acid used as the catalyst, filtering the precipitant, and concentrating the residual solution by vacuum. The concentrated solution is then filtered, washed with solvent, and concentrated by vacuum again to obtain compound (III).

Scheme 3, below, illustrates a third step in the process of the present invention wherein intermediate compound (IV) is synthesized.

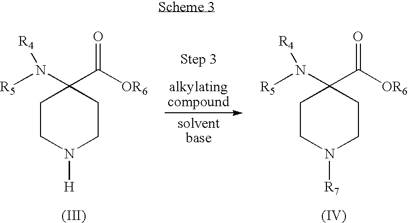

In Scheme 3, compound (III) is mixed in with an alkylating agent in the presence of a solvent and a base to form intermediate compound (IV), wherein R₇ is hydrocarbyl or substituted hydrocarbyl. Preferably, R₇ is selected from the group consisting of R₂₁OC(O)R₂₂—, R₂₁C(O)OR₂₂—, R₂₁OR₂₃OC(O)R₂₂—, R₂₄R₂₂—, and R₂₅R₂₂—, wherein R₂₁, R₂₂, and R₂₃ are independently hydrocarbyl or substituted hydrocarbyl, R₂₄ is cycloalkyl or substituted cycloalkyl, and R₂₅ is heterocyclic. Preferably, R₂₁, R₂₂, and R₂₃ are independently alkyl, alkoxy, alkenyl, and alkenyloxy groups, R₂₄ is a 5- to 7-member cycloalkyl, and R₂₅ is a 5- to 7-member heterocyclic; more preferably, R₂₁, R₂₂, and R₂₃ are independently linear or branched alkyl, alkoxy, alkenyl, and alkenyloxy groups having about 1 to about 18 carbon atoms, R₂₄ is a 5- to 7-member cycloalkyl, and R₂₅ is a 5- to 7-member heterocyclic comprising 1 to 5 hetero-atoms selected from oxygen, sulfur, and nitrogen; still more preferably, R₇ is methyl propionate, ethyl propionate, 2-phenylethyl, 2-(2-thienyl)ethyl, and 2-(4-ethyl-4,5-dihydro-5-oxo-1H-tetrazol-1-yl)ethyl.

General examples of alkylating agents include compounds having the structure:

L-R₂₆-R₂₇

wherein L is a displacement or leaving group. In one embodiment, L, R₂₆, and R₂₇ are independently hydrocarbyl or substituted hydrocarbyl. Preferably, L is a halide, toluenesulfonate, or methylsulfonate; R₂₆ is a hydrocarbyl or substituted hydrocarbyl group having 1 to 18 carbons; and R₂₇ is selected from R₂₁OC(O)R₂₂—, R₂₁C(O)OR₂₂—, R₂₁OR₂₃OC(O)R₂₂—, R₂₄R₂₂—, and R₂₅R₂₂—, wherein R₂₁, R₂₂, R₂₃, R₂₄, and R₂₅, are as defined above; preferably, L is a halide, toluenesulfonate, or methylsulfonate, R₂₆ is ethyl, and R₂₇ is —C(O)OCH₃, —C(O)OCH₂CH₃, -phenyl, -2-(2-thienyl), and -2-(4-ethyl-4,5-dihydro-5-oxo-1H-tetrazol-1-yl)ethyl.

The alkylating agents can also comprise an electron deficient moiety to an electron withdrawing group such as carbonyl, nitrile, carbonyl-oxy, alkyl carbonate, and alkyl-alkoxy carbonate. Some specific examples of the alkylating agents include methyl acrylate, ethyl acrylate, acrylic acid, acrylonitrile, acrylamide, acrolein, phenylethyl halide, tolylate, mesylate, styrene, and substituted styrene. An illustration of alkylating agents comprising an electron deficient moiety is as follows:

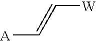

wherein A is hydrogen, hydrocarbyl, or substituted hydrocarbyl and W is hydrocarbyl, substituted hydrocarbyl, nitrile, and amide. In one example, A is hydrogen, an alkyl comprised of 1 to 18 carbons, aryl, substituted aryl, alkylaryl wherein the alkyl group is comprised of 1 to 18 carbons, and a hydrocarbyl or substituted hydrocarbyl 5- to 7-member ring; and W is carboxylic acid, carboxylic acid ester, nitrile, amide, carbonyl, or aryl. Most preferably A is hydrogen and W is a carboxylic acid ester or aryl.

Examples of the base used in the reaction of Scheme 3 include metal hydroxide, metal alkoxide, metal hydride, metal carbonate, metal hydrogen carbonate, amine, quaternary alkyl ammonia hydroxide, and ammonia. Examples of metal alkoxides and metal hydrides include sodium, potassium, cesium, magnesium, aluminum alkoxides and hydrides and the like. Preferably, the base is ammonia or a metal alkoxide.

The solvent of Scheme 3 is an organic solvent. Preferred solvents include, dimethyl sulfoxide, ether, dichloromethane, chloroform, carbon tetrachloride, ethylene chloride, acetonitrile, toluene, ethylacetate, propylacetate, butylacetate, alcohol ethers, alkanols containing 1 to 18 carbon atoms, hydrocarbons containing 1 to 18 carbon atoms, aryl-alcohol, and 5 to 7 member heterocyclic alcohols comprising 1 to 5 hetero-atoms selected from oxygen, sulfur, and nitrogen which may or may not contain heteroatoms like O, S and N. Most preferable solvents are acetonitrile, chloroform, 1,2-dichloroethane, 1,1,2-trichloroethane, dichloromethane, and carbon tetrachloride.

In one embodiment, the reaction mixture comprises about 1 molar equivalent to about 5 molar equivalents of alkylating agent and about 1 molar equivalent to about 5 molar equivalents of base per molar equivalent of compound (III). Preferably, the reaction mixture is charged with about 1 to about 3 equivalents of an alkylating agent and about 1 equivalent to about 3 equivalents of base per molar equivalent of compound (III).

The solvent to compound (III) ratio on a volume to weight basis is about 1:2 to about 1:100, preferably, the solvent to compound ratio is about 1:4 to about 1:50.

In one embodiment, the temperature of the reaction mixture during the reaction ranges from about −10° C. to about 65° C. In another embodiment, the reaction temperature ranges from about 10° C. to about 40° C. The reaction mixture is permitted to react up to a couple of days. In one example, the reaction is carried out up to about 24 hours. In another example, the reaction time is from about 2 hours to about 6 hours.

In one embodiment, methyl acrylate was added to compound (III) dispersed in methanol, and triethylamine was added and mixed for 1 hour. The resulting solid was filtered off and the methanolic solution concentrated by vacuum to obtain compound (IV). Compound (IV) may be further purified through recrystallization with organic solvents, preparative chromatography or a combination of methods.

Scheme 4, below, illustrates a fourth step in the process of the present invention in which compound (V) is synthesized.

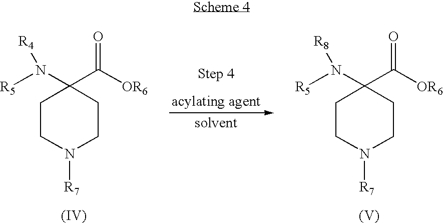

In Scheme 4, compound (IV) is reacted with an acylating agent in a reaction mixture containing a solvent to form compound (V), wherein R₈ is an acyl moiety corresponding to the acylating agent. Compound (V) may be an analgesic, for example remifentanil or carfentanil, or an intermediate used in the production of synthetic opiate or opioid compounds.

The temperature of the reaction mixture ranges from about 20° C. to about 80° C. In another example, the reaction temperature ranges from about 40° C. to about 65° C. The reaction mixture is permitted to react from about 4 hours to about 18 hours. In one example, the reaction is carried out from about 4 hours to about 8 hours.

In one embodiment, R₈ is —CO—R₉ and R₉ is hydrocarbyl or substituted hydrocarbyl. In another example, the acylating agent is an acid halide, preferably a C₁-C₁₈ acid halide selected from alkyl acid halides and alkoxy-alkyl halides. Examples of acylating agents include, but are not limited to, acetyl chloride, propionyl chloride, propionic anhydride, methyl ketene, butanoyl chloride, alkyl acid cyanides, and the like. In one embodiment, the alkyl group contains between 1 to 18 carbons. In another embodiment, the alkyl group contains between 2 to 4 carbons. Preferably the acylating agent is propionyl chloride or propionic anhydride.

The solvent contained in the reaction mixture can be any solvent that is inert to the reaction occurring in Scheme 4. Examples of such solvents include, but are not limited to acetonitrile; acetone; dichloromethane; chloroform; n,n-dimethylformamide; dimethylsulfoxide; ethylacetate; dichloroethane; aromatic hydrocarbons such as benzene, toluene, and xylene; lower alkanol such as methanol, ethanol, isopropanol, n-propanol, 1-butanol, tert-butanol, and the like; ketones such as 4-methyl-2-pentanone and the like; ethers such as 1,4-dioxane, tetrahydrofuran (THF), 1,1-oxybisethane, and the like; nitrobenzene; and mixtures thereof. In one example, the reaction mixture contains acetonitrile.

The reaction mixture optionally contains an acid scavenger. The acid scavenger may include metal hydrides, hydroxides, carbonates, bicarbonates, amines, and the like.

In one embodiment, the reaction mixture comprises about 1 molar equivalents to about 50 molar equivalents of acylating agent per molar equivalent of compound (IV). Preferably, the reaction mixture is charged with about 2 to about 5 molar equivalents of an acylating agent per molar equivalent of compound (IV). The solvent to compound (IV) ratio on a volume to weight basis is about 1:4 to about 1:50, preferably, the solvent to compound ratio is 1:4 to 1:25. Compound (V) is collected by filtration and drying. The product may be purified by methods known in the art including recrystallization and/or solvent extraction. The overall process of the present invention for synthesizing opiate or opioid analgesics and anesthetics that incorporates the individual steps described above is illustrated in Scheme 5, below.

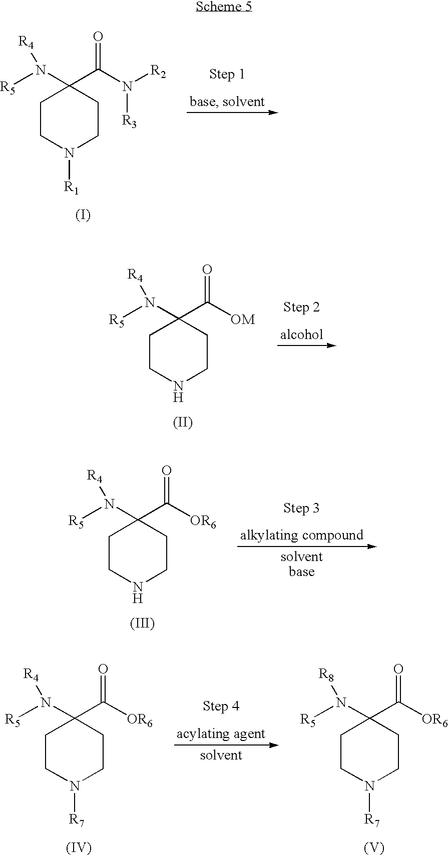

The process of the present invention described above significantly improves the synthesis reactions for producing analgesics or anesthetics by reducing the overall number of steps and eliminating the use of cyanide compounds. The ability to convert the amide group of compound (I) to a carboxylic acid functional group on compound (II) greatly improves the synthesis process by enabling esterification of compound (II) without the use of potentially hazardous cyanide compounds. In the process of synthesizing remifentanil and carfentanil the hydrolysis and esterification reaction is illustrated in Scheme 6.

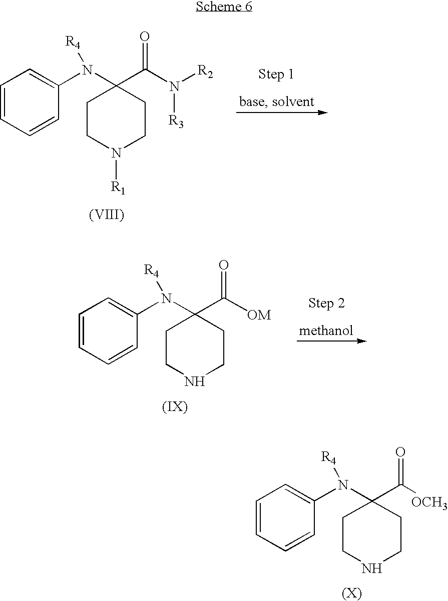

In the synthesis of remifentanil or carfentanil, R₄ is preferably hydrogen.

In one embodiment of the present invention, a process for synthesizing remifentanil is provided. An illustration of this process is shown below in Scheme 7.

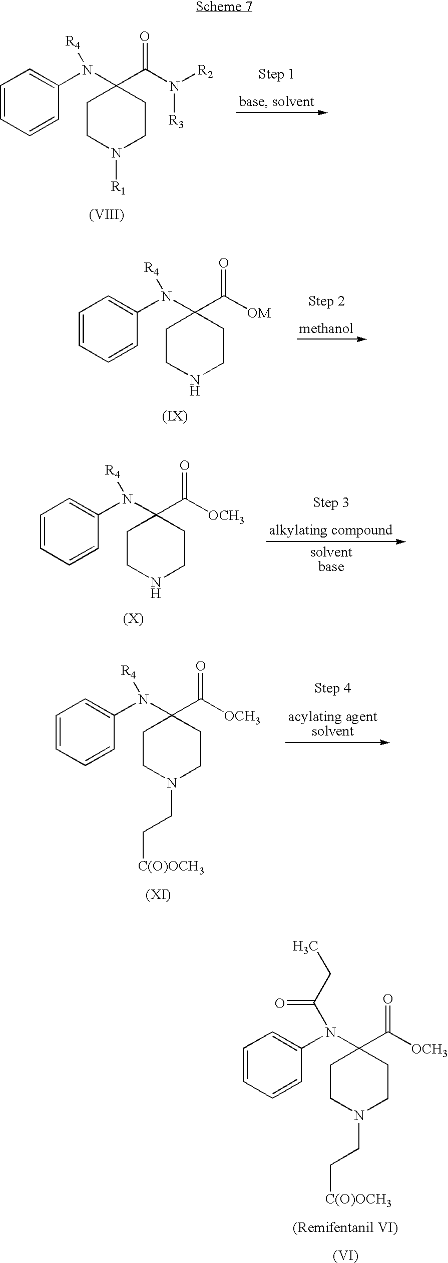

Compound (VIII), for example, 1-(carbethoxy)-4-(phenylamino)-4-piperidinecarboxanilide, is reacted with a base in the presence of a solvent in Step 1. Preferably the reaction occurs under elevated temperature, more preferably, the reaction occurs under elevated temperature and pressure which may be obtained by conducting the reaction in a closed reaction vessel heated to produce an increased vapor pressure within the vessel.

In one embodiment the reaction was carried out at an elevated temperature range from about 120° C. to about 200° C. In one embodiment the reaction ranges from about 145° C. to about 175° C. The residual pressure within the closed reaction chamber ranges from about 65 p.s.i to about 165 p.s.i. In one embodiment, the reaction was carried out at 145° C. in a closed reaction vessel, for example a Hastelloy C Parr reactor, resulting in a residual pressure of 165 p.s.i. In another example, the reaction was carried out at 175° C. in a Hastelloy 276 Parr reactor and resulted in a residual pressure of 150 p.s.i.

The reaction mixture is permitted to react up to a couple days. In one embodiment, the reaction is carried out up to about 28 hours. In another example, the reaction time is from about 4 hours to about 18 hours.

In one embodiment, the reaction mixture comprises about 1 equivalent to about 6 equivalents of base per equivalent of compound (VIII). The solvent to compound (VIII) weight ratio is about 1:2 to about 1:100.

The reaction mixture contains a base; the base hydrolyzes the amide group and may be any base capable of such hydrolysis. Preferably, the base is a strong base, more preferably the base is a metal hydroxide such as sodium hydroxide.

The solvent used in the reaction mixture can include water and/or one or more organic solvents. Examples of solvents include, but are not limited to benzene, toluene, xylene, methanol, ethanol, isopropanol, n-propanol, 1-butanol, tert-butanol, 1,4-dioxane, tetrahydrofuran (THF), and 1,1-oxybisethane. In one embodiment the solvent is isopropanol or methanol. The hydrolysis step results in compound (IX). In one embodiment the product is isolated by filtration and vacuum drying. In another example the product is isolated by the addition of acid to the reaction mixture to dissolve the product, the solution is filtered, and the product precipitated by the addition of base, the precipitate is then filtered, washed, and dried.

In Step 2, compound (IX), for example, N-phenyl-α-(4-piperidino)glycine, is reacted in a reaction mixture with methanol to form compound (X). The reaction may optionally be carried out in the presence of a catalyst and/or desiccant.

In one embodiment, the reaction mixture comprises about 2 molar equivalents to about 100 molar equivalents of methanol per molar equivalent of compound (IX). In another embodiment, the reaction mixture is charged with about 4 molar equivalents to about 50 molar equivalents of methanol per molar equivalent of compound (IX).

The temperature of the reaction mixture during the reaction ranges from about 25° C. to about 80° C. In another example, the reaction temperature ranges from about 50° C. to about 70° C. The reaction mixture is permitted to react up to a few days. In one example, the reaction is from about 8 to about 100 hours. Preferably, the reaction time is from about 24 hours to about 60 hours.

Desiccant can be used to enhance the rate of esterification of compound (IX). Non-limiting examples of desiccant compounds include trimethyl orthoformate, sulfur trioxide, polyphosphoric acid, phosphorous pentoxide, molecular sieves, alumina, silica gel, sodium sulfate anhydrous, magnesium sulfate, and the like. In one embodiment, the desiccant is trimethyl orthoformate. In one example the reaction mixture is charged with about 1 molar equivalent to about 5 molar equivalents of desiccant, per molar equivalent of compound (IX), preferably 1 molar equivalent to about 3 molar equivalents of desiccant per molar equivalent of compound (IX).

The catalyst can be selected from the group commonly known as Bronsted acids or Lewis acids. In one embodiment the catalyst is sulfuric acid. In one embodiment the reaction mixture comprised about 1 molar equivalent to about 10 molar equivalents of the catalyst per molar equivalent of compound (IX).

In one embodiment, compound (X) is isolated by neutralizing the reaction with solid sodium carbonate and water, followed by solvent extraction with ethyl acetate which is then separated, air dried, and re-dissolved in methanol to yield purified compound (X) in the methanol solution. In another embodiment, compound (X) is isolated by cooling the reaction to below 10° C., adding triethylamine to precipitate the resulting anion of an appropriate Bronsted acid used as the catalyst, filtering the precipitant, and concentrating the residual solution by vacuum. The concentrated solution is then filtered, washed with solvent, and concentrated by vacuum again to obtain compound (X).

In step 3, compound (X), for example, N-phenyl-α-(4-piperidino)glycine methyl ester, is mixed with methyl acrylate (alkylating agent), in the presence of a solvent and a base to form compound (XI).

In one embodiment, the reaction mixture comprises about 1 molar equivalent to about 5 molar equivalents of alkylating agent and about 1 molar equivalent to about 5 molar equivalents of base per molar equivalent of compound (X). Preferably, the reaction mixture is charged with about 1 to about 3 molar equivalents of alkylating agent and about 1 to about 3 molar equivalents of base per molar equivalent of compound (X). The solvent to compound (X) ratio on a weight to volume basis is about 1:2 to 1:100, preferably, the solvent to compound ratio is 1:4 to 1:50.

The temperature of the reaction mixture during the reaction ranges from about −10° C. to about 65° C. In another embodiment, the reaction temperature ranges from about 10° C. to about 40° C. The reaction mixture is permitted to react up to a couple of days. In one example, the reaction is carried out about 24 hours. In another example, the reaction time is from about 2 hours to about 6 hours.

Preferred solvents are acetonitrile, chloroform, 1,2-dichloroethane, 1,1,2-trichloroethane, dichloromethane, carbon tetrachloride, and methanol

In one embodiment, methyl acrylate was added to compound (X) dispersed in methanol, and triethylamine was added and mixed for 1 hour. The resulting solid was filtered off and the methanolic solution concentrated by vacuum to obtain compound (XI). Compound (XI) may be further purified through recrystallization with organic solvents, preparative chromatography, or a combination of methods.

In Step 4, compound (XI), for example, methyl 3-(4-anilino-4-carbomethoxy-piperidino) propionate, is reacted with an acylating agent in a reaction mixture containing a solvent to form remifentanil (compound (VI)).

Preferably the acylating agent is propionyl chloride or propionic anhydride.

The temperature of the reaction mixture ranges from about 20° C. to about 80° C. In another example, the reaction temperature ranges from about 40° C. to about 65° C. The reaction mixture is permitted to react from about 4 hours to about 18 hours, preferably from about 4 hours to about 8 hours.

The solvent contained in the reaction mixture can be any solvent that is inert to the reaction occurring in Step 4. Examples of such solvents include, but are not limited to acetonitrile; acetone; dichloromethane; chloroform; n,n-dimethylformamide; dimethylsulfoxide; ethylacetate; dichloroethane; aromatic hydrocarbons such as benzene, toluene, and xylene; and the like; ketones such as 4-methyl-2-pentanone and the like; ethers such as 1,4-dioxane, tetrahydrofuran (THF), 1,1-oxybisethane, and the like; nitrobenzene; and mixtures thereof. In one example, the reaction mixture contains acetonitrile.

In one embodiment, the reaction mixture comprises about 1 molar equivalent to about 50 molar equivalents of acylating agent per molar equivalent of compound (IV). Preferably, the reaction mixture is charged with about 2 to about 5 molar equivalents of an acylating agent per molar equivalent of compound (IV). The solvent to compound (XI) ratio on a volume to weight basis is about 1:4 to about 1:25, preferably, the solvent to compound ratio is 1:4 to 1:15.

Remifentanil is collected by filtration and drying. The product may be purified by recrystallization, solvent extraction, or any other methods or combination of methods known in the art.

In another embodiment of the present invention, a process for synthesizing carfentanil, compound (VII) is provided. This process is illustrated below in Scheme 8.

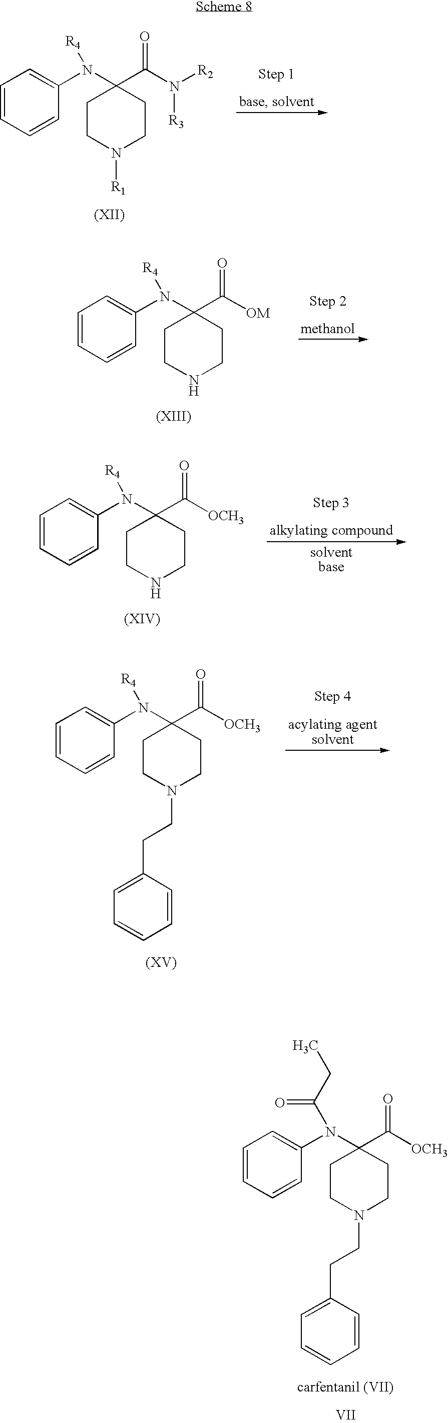

The method of producing carfentanil is nearly identical to that of remifentanil with the exception of Step 3 wherein the alkylating compound used to produce carfentanil is styrene. Other than the substitution of styrene for methyl acrylate as the alkylating compound of Step 3, the reaction conditions of Scheme 7, as described above, are identical to those of Scheme 8.

Preferred embodiments of compound (I) for schemes 1, 5, 6, 7 and 8 are illustrated below as compounds XVI, XVII, XVIII, and XIX.

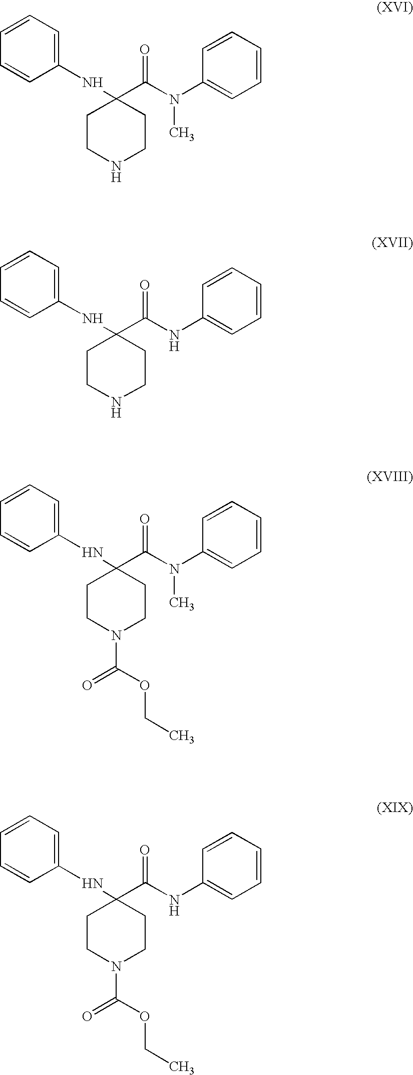

The preferred embodiment of compound (II) for Schemes 2, 5, 6, 7, and 8 is N-phenyl-α-(4-piperidino)glycine, having the formula (XX), illustrated below.

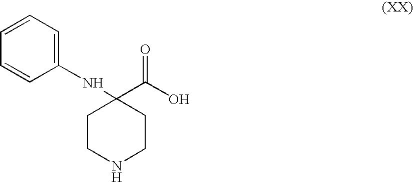

The product compounds synthesized according to the process of the present invention may be used as synthetic opiates or opioids for analgesic or anesthetic purposes. In particular, the remifentanil compounds of the present invention can be used as anesthetics in surgical procedures wherein the compounds have a beneficially short half-life in humans that permit patients to awaken shortly after a surgical procedure has been concluded.

### EXAMPLES

The following examples are provided in order to more fully illustrate the present invention.

### Examples of Step 1

### Example 1

A solution of compound (XIX) (10 g), 50% sodium hydroxide (10 g), and isopropanol (100 mL), was stirred at 155° C./70 p.s.i. for 4 days in a 450 mL Hastelloy 276 Parr reactor. After the reaction was allowed to cool to room temperature, the resulting solid was filtered and washed with isopropanol and then with ether under suction to obtain 8 g white powder identified by mass spectrometry as N-phenyl-α-(4-piperidino)glycine.

### Example 2

A solution of compound (XVII) (2 g), solid potassium hydroxide (4 g), and methanol (100 mL) was heated in a 450 mL Hastelloy C Parr reactor at 145° C. for 4 hours at a residual pressure of 165 p.s.i. The reaction was then cooled to ambient temperature and the internal pressure was relieved. The reaction solution was analyzed by LC-MS; the major product had a [M+H]⁺ peak at m/z 296 which represented compound (XVII) and a trace amount of product with a [M+H]⁺ peak at m/z 221 which represented compound (XX). The reaction was continued under the same conditions for another 24 hours resulting in a higher yield of N-phenyl-α-(4-piperidino)glycine.

### Example 3

A solution of compound (XVII) (0.5 g), 50% sodium hydroxide (10 mL), and ethanol (100 mL) was heated in a 450 mL Hastelloy C Parr reactor at 145° C. for 4 hours at a residual pressure of 65 p.s.i. The reaction was then cooled to ambient temperature and the internal pressure relieved. The reaction solution was analyzed by LC-MS which found the major product had a [M+H]⁺ peak at m/z 296 which represented compound (XVII) and a good amount of product with a [M+H]⁺ peak at m/z 221 which represented compound (XX). The reaction was continued at 175° C./150 p.s.i. for another 24 hours and resulted in a higher conversion to product with a [M+H]⁺ peak at m/z 221 identified as N-phenyl-α-(4-piperidino)glycine.

### Example 4

A solution of compound (XVI) (1 g), 50% sodium hydroxide (10 mL), and isopropanol (100 mL) was heated in a 450 mL Hastelloy C Parr reactor at 145° C. for 20 hours at a residual pressure of 65 p.s.i. The reaction was then cooled to ambient temperature and the internal pressure was relieved. The reaction solution was analyzed by LC-MS and found a major product had a [M+H]⁺ peak at m/z 221 which represented N-phenyl-α-(4-piperidino)glycine and a good amount of product with a [M+H]⁺ peak at m/z 108 which represented N-methylaniline.

### Example 5

A solution of compound (XVI) (5 g), 50% sodium hydroxide (2 g), and isopropanol (50 mL) was heated in a 450 mL Hastelloy C Parr reactor at 155° C. for 20 hours at a residual pressure of 65 p.s.i. The reaction was cooled to ambient temperature and the internal pressure relieved. The solid was filtered and dried under negative pressure to obtain 3.23 g white powder identified by mass spectrometry as N-phenyl-α-(4-piperidino)glycine.

### Example 6

A solution of compound (XIX) (100 g), sodium hydroxide pellets (48.75 g), and isopropanol (250 mL) was heated in a 450 mL Hastelloy C Parr reactor at 155° C. for 18 hours at a residual pressure of 65 p.s.i. The reaction was then cooled to ambient temperature and the internal pressure was relieved. The reaction slurry was poured into a one liter beaker. The reactor was rinsed with 200 mL of water to dissolve any remaining solids in the reactor. The water rinse was added to the beaker. The solid in the slurry dissolved with the addition of concentrated hydrochloric acid. Enough hydrochloric acid was added to adjust the pH to about 2-3. The solution was then made alkaline with 50% sodium hydroxide. Precipitation occurred and the solid was filtered, washed with water, and dried at 100° C. in a vacuum oven over 3 days to obtain 60 g white powder identified as N-phenyl-α-(4-piperidino)glycine.

### Example 7

A solution of compound (XIX) (80 g; 92.5% assay; 0.2 mole), ethanol (200 mL), and sodium hydroxide pellets (39 g; 0.975 mole) was placed in a Parr stirred pressure reactor. The closed reactor was purged with nitrogen and heated to 155° C. The resulting pressure was 110-120 p.s.i. The reaction solution was stirred at this temperature and pressure for 18 hours. The heat was removed and the reaction vessel was allowed to cool to about 50° C. The residual pressure was released by opening the reactor. The reaction slurry was poured into a one liter beaker. The reactor was rinsed with 200 mL of water to dissolve any remaining solids in the reactor. The water rinse was added to the beaker. The pH of the solution in the beaker was adjusted to about pH 1.5 by the addition of hydrochloric acid while the temperature of the solution was controlled at 20-40° C. by the slow addition of the acid and a cooling bath. A clear, light brown solution was obtained. About 120 mL of hydrochloric acid was required. During the acid addition, carbon dioxide was liberated from the solution. The pH was adjusted to about 7-9 by the slow addition of 25% sodium hydroxide solution. Solid precipitated during the base addition. The resulting slurry was cooled to 0-10° C. and stirred for one hour. The slurry was filtered and the cake washed with 50 mL of water and then washed twice with 50 mL of ethanol. The ethanol washes removed the brown color from the solid and made the solid easier to dry. The solid was dried overnight at 75° C. The reaction yielded 42.4 g of N-phenyl-α-(4-piperidino)glycine.

### Example 8

A solution of compound (XIX) (50 g), sodium hydroxide pellets (16.25 g), and isopropanol (250 mL) was poured into a 450 mL Hastelloy 276 Parr reactor and stirred at 155° C./70 p.s.i. overnight. The reaction was allowed to cool to room temperature, the resulting solid was filtered and washed with isopropanol, washed with ethanol and washed with ether under suction to obtain 34.9 g white powder identified by liquid chromatography as N-phenyl-α-(4-piperidino)glycine.

### Example 9

A solution of compound (XIX) (100 g), sodium hydroxide (48.8 g), and ethanol grade 3A (250 mL) was poured into a 450 mL Hastelloy 276 Parr reactor which was then purged with nitrogen and stirred at 155° C. 1115 p.s.i. for 18 hours. After the reaction was cooled to 50° C., the contents were transferred to a 1 liter beaker and the reactor was rinsed with 250 mL water which was then added to the beaker. Enough concentrated hydrochloric acid was added drop-wise to adjust the pH to about 1.5. All solid dissolved. The solution was filtered and the pH adjusted to about 9-10 with 25% sodium hydroxide solution. Solid precipitated during the base addition. The precipitated solid was filtered and washed with methanol followed by drying at 75° C. to obtain 56.8 g white solid identified by liquid chromatography as N-phenyl-α-(4-piperidino)glycine.

### Example 10

A solution of compound (XVI) (20 g), 50% sodium hydroxide (10 g), and isopropanol (200 mL) was poured into a 450 mL Hastelloy 276 Parr reactor and stirred at 155° C./70 p.s.i. for 1 day. After the reaction was cooled to room temperature, the solid was filtered and washed with isopropanol and then washed with ether under suction to obtain 11.32 g white powder identified by liquid chromatography as N-phenyl-α-(4-piperidino)glycine.

### Example of Step 2

### Example 11

N-phenyl-α-(4-piperidino)glycine (20 g) was suspended in methanol (350 mL) by stirring. Sulfuric acid (30 mL) was slowly added to the suspension such that the temperature of the suspension remained below 65° C. The reaction solution was stirred at 65° C. overnight. The solution was allowed to cool to below 10° C. in an ice bath. Triethylamine (60 mL) was added drop wise such that the solution maintained a temperature below 20° C. Solid precipitated out of solution. The solid (triethylammonium sulfate) was filtered under suction. The residual solution was concentrated in vacuum and 100 g of amber oil was obtained. The oil was filtered through 100 g of silica gel by washing it with methylene chloride (1000 mL). The methylene chloride solution was concentrated under vacuum to obtain 49 g of light brown oil identified by mass spectrometry as N-phenyl-α-(4-piperidino)glycine methyl ester.

### Examples of Steps 2 & 3

### Example 12

N-phenyl-α-(4-piperidino)glycine sodium salt (15 g) was suspended in methanol (150 mL) with stirring. Sulfuric acid (9 mL) was slowly added such that the reaction mixture was maintained at a temperature below 65° C. The reaction solution was stirred at 65° C. for 3 days. Solid sodium bicarbonate (10 g) was added followed by about 30 ml water. The product was extracted into ethyl acetate. The ethyl acetate solution was separated and air dried. The resulting residue was dispersed into methanol. The methanol solution contained exclusively the desirable product, identified by mass spectrometry as N-phenyl-α-(4-piperidino)glycine methyl ester. The methanol solution was cooled below 10° C. and methyl acrylate (6 g) was added slowly to maintain the temperature below 40° C. The solution was stirred for 30 minutes then the solution was cooled to room temperature. Triethylamine (20 mL) was added and stirred for 1 hour. The resulting solid was filtered off and the methanolic solution was concentrated under vacuum. The product was identified by mass spectrometry as methyl 3-(4-anilino-4-carbomethoxy-piperidino) propionate.

### Example 13

Sulfuric acid (55 g) was slowly added to a suspension of N-phenyl-α-(4-piperidino)glycine sodium salt (40 g), methanol AR (750 mL), and trimethyl orthoformate (250 mL) such that the suspension temperature remained below 45° C. The suspension was stirred at reflux for 48 hours and was monitored by HPLC. The reaction was cooled to 15-25° C. and 25% sodium methoxide in methanol (120 mL) was added to the suspension while the temperature of the suspension was cooled to 10-15° C. Methyl acrylate (20 mL) was added to the suspension which was allowed to warm to room temperature, then stirred at 40° C. for 1 hour, and cooled to room temperature. The resulting solid was filtered off and washed with methanol (100 mL). The remaining solution was concentrated, water (500 mL) added, and the product extracted with dichloromethane (100 mL). The aqueous phase was washed with dichloromethane (50 mL). The dichloromethane solutions were combined and dried over magnesium sulfate and concentrated under vacuum to obtain 40.4 g of pink solid. The pink solid was re-dissolved into 250 mL dichloromethane and filtered through a funnel containing silica gel (70 g) the product eluted with 2 liters of ethyl acetate. The ethyl acetate solution was evaporated under vacuum to dryness to obtain 35 g of white solid identified as methyl 3-(4-anilino-4-carbomethoxy-piperidino) propionate.

### Examples of Step 4

### Example 14

Methyl 3-(4-anilino-4-carbomethoxy-piperidino) propionate (1 g) was dissolved into 25 mL of acetonitrile. Propionyl chloride (1 mL) was added to the solution which was then stirred at 65° C. for 18 hours. Precipitation occurred. The solution was concentrated under vacuum and 50 mL of acetone was added to the reaction mass to disperse the product. The solid was filtered under suction to obtain 0.8 g white solid characterized by HPLC-UV as remifentanil hydrochloride.

### Example 15

Methyl 3-(4-anilino-4-carbomethoxy-piperidino) propionate (90 mg) was dissolved into 5 mL of anhydrous acetonitrile. About 0.03 mL of propionyl chloride was added to the solution which was then stirred for 1 hour. After 1 hour LC-UV at λ210 nm showed about 60% conversion to remifentanil. The solution was stirred for another four hours at room temperature which resulted in about 70% conversion. The solution was heated to 50° C. and allowed to react for two days. After two days an aliquot of the solution was subjected to LC-MS and LC-UV analysis which showed almost complete conversion to remifentanil with some impurities detected at λ254 nm. The solution was neutralized with a saturated solution of sodium bicarbonate. The product was extracted with ethylacetate. The ethylacetate solution was dried over magnesium sulfate and concentrated in vacuo to obtain a light yellow oil. The oil was dissolved into 2 mL of isopropyl alcohol to which concentrated hydrochloric acid (0.5 mL) was added. Precipitation occurred and the solution was cooled in the refrigerator for 1 hour before filtration to obtain 0.05 g (43% yield) of an off-white powder. This powder was analyzed by LC-UV and identified as remifentanil.

### ABBREVIATIONS AND DEFINITIONS

The term “acyl” is a radical provided by the residue after removal of hydroxyl from an organic acid, for example, COOH of an organic carboxylic acid, e.g., RC(O)—, wherein R is R₂₈, R₂₈O—, R₂₈R₂₉N—, or R₂₉S—, R₂₈ is hydrocarbyl, heterosubstituted hydrocarbyl, or heterocyclo and R₂₉ is hydrogen, hydrocarbyl or substituted hydrocarbyl. Examples of such acyl radicals include alkanoyl and aroyl radicals. Examples of lower alkanoyl radicals include formyl, acetyl, propionyl, butyryl, isobutyryl, valeryl, isovaleryl, pivaloyl, hexanoyl, and trifluoroacetyl.

The term “alkenyl” is a linear or branched radical having at least one carbon-carbon double bond of two to about twenty carbon atoms or, preferably, two to about twelve carbon atoms. More preferred alkyl radicals are “lower alkenyl” radicals having two to about six carbon atoms. Examples of alkenyl radicals include ethenyl, propenyl, allyl, butenyl and 4-methylbutenyl. The terms “alkenyl” and “lower alkenyl” also are radicals having “cis” and “trans” orientations, or alternatively, “E” and “Z” orientations.

The term “cycloalkyl” or “cyclic alkyl” is a saturated carbocyclic radical having three to twelve carbon atoms. More preferred cycloalkyl radicals are “lower cycloalkyl” radicals having three to about eight carbon atoms. Examples of such radicals include cyclopropyl, cyclobutyl, cyclopentyl and cyclohexyl.

The term “substituted cycloalkyl” or “substituted cyclic alkyl” is a cycloalkyl in which one or more hydrogen atom to any carbon of the cycloalkyl is replaced by another group. The group may be a halogen, alkyl, substituted alkyl, aryl, substituted aryl, heteroaryl, substituted heteroaryl, alkoxy, aryloxy, amino, silyl, thio, and combinations thereof. Examples of such radicals include, bromocyclohexyl and methyl cyclopentyl.

The terms “alkoxy” and “alkyloxy” are linear or branched oxy-containing radicals each having alkyl portions of one to about ten carbon atoms. More preferred alkoxy radicals are “lower alkoxy” radicals having one to six carbon atoms. Examples of such radicals include methoxy, ethoxy, propoxy, butoxy and tert-butoxy.

The term “alkoxyalkyl” is an alkyl radical having one or more alkoxy radicals attached to the alkyl radical, that is, to form monoalkoxyalkyl and dialkoxyalkyl radicals. The “alkoxy” radicals may be further substituted with one or more halo atoms, such as fluoro, chloro or bromo, to provide haloalkoxy radicals. More preferred haloalkoxy radicals are “lower haloalkoxy” radicals having one to six carbon atoms and one or more halo radicals. Examples of such radicals include fluoromethoxy, chloromethoxy, trifluoromethoxy, trifluoroethoxy, fluoroethoxy and fluoropropoxy.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “amino” as used herein alone or as part of another group denotes the moiety —NR₃₀R₃₁ wherein R₃₀ and R₃₁ are independently hydrocarbyl, substituted hydrocarbyl or heterocyclo.

The terms “halide,” “halogen,” or “halo” as used herein alone or as part of another group refer to chlorine, bromine, fluorine, and iodine.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring, preferably 5 to 7 atoms in each ring with 1 to 5 hetero atoms selected from oxygen, sulfur, and nitrogen. More preferably, the heterocyclo group has 1 or 2 oxygen atoms, 1 or 2 sulfur atoms, and/or 1 to 4 nitrogen atoms in the ring, and may be bonded to the remainder of the molecule through a carbon or heteroatom. Exemplary heterocyclo include heteroaromatics such as furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The term “heteroaromatic” as used herein alone or as part of another group denote optionally substituted aromatic groups having at least one heteroatom in at least one ring, and preferably 5 or 6 atoms in each ring. The heteroaromatic group preferably has 1 or 2 oxygen atoms, 1 or 2 sulfur atoms, and/or 1 to 4 nitrogen atoms in the ring, and may be bonded to the remainder of the molecule through a carbon or heteroatom. Exemplary heteroaromatics include furyl, thienyl, pyridyl, oxazolyl, pyrrolyl, indolyl, quinolinyl, or isoquinolinyl and the like. Exemplary substituents include one or more of the following groups: hydrocarbyl, substituted hydrocarbyl, keto, hydroxy, acyl, acyloxy, alkoxy, alkenoxy, alkynoxy, aryloxy, halogen, amido, amino, nitro, cyano, thiol, ketals, acetals, esters and ethers.

The terms “hydrocarbon” and “hydrocarbyl” as used herein describe organic compounds or radicals consisting exclusively of the elements carbon and hydrogen. These moieties include alkyl, alkenyl, alkynyl, and aryl moieties. These moieties also include alkyl, alkenyl, alkynyl, and aryl moieties substituted with other aliphatic or cyclic hydrocarbon groups, such as alkaryl, alkenaryl and alkynaryl. Unless otherwise indicated, these moieties comprise 1 to 18 carbon atoms. They may be straight or branched chain or cyclic and include methyl, ethyl, propyl, isopropyl, allyl, benzyl, hexyl and the like.

The “substituted hydrocarbyl” moieties described herein are hydrocarbyl moieties which are substituted with at least one atom other than carbon, including moieties in which a carbon chain atom is substituted with a hetero atom such as nitrogen, oxygen, silicon, phosphorous, boron, sulfur, or a halogen atom. These substituents include halogen, heterocyclo, alkoxy, alkenoxyl alkynoxy, aryloxy, hydroxy, keto, acyl, acyloxy, nitro, tertiaryamino, amido, nitro, cyano, ketals, acetals, esters and ethers.

When introducing elements of the present invention or the preferred embodiment(s) thereof, the articles “a,” “an,” “the,” and “said” are intended to mean that there are one or more of the elements. The terms “comprising,” “including,” and “having” are intended to be inclusive and mean that there may be additional elements other than the listed elements.

In view of the above, it will be seen that the several objects of the invention are achieved and other advantageous results attained. As various changes could be made in the above methods and products without departing from the scope of the invention, it is intended that all matter contained in the above description and shown in any accompanying drawings shall be interpreted as illustrative and not in a limiting sense.

What is claimed is:

1-36. (canceled)

37. A process for the preparation of an opiate or opioid analgesic or anesthetic, comprising:
reacting a compound (I) having the formula: 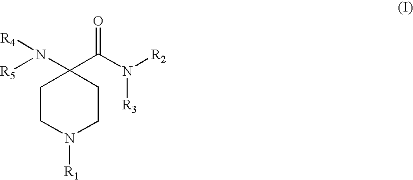
with a base the presence of a solvent to form intermediate compound (II) having the formula: 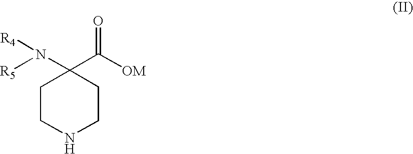
wherein R₁, R₂, R₃, R₄, and R₅ are independently selected from the group consisting of hydrogen, hydrocarbyl, and substituted hydrocarbyl and M is hydrogen or a cation;
reacting the intermediate compound (II) with an alcohol, R₆OH, to form an intermediate compound (III): 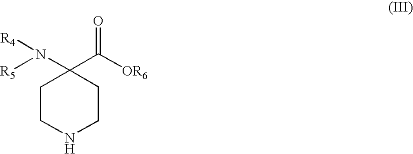
wherein R₆ is selected from the group consisting of hydrocarbyl and substituted hydrocarbyl;
reacting the intermediate compound (III) with an alkylating agent to form an intermediate compound (IV): 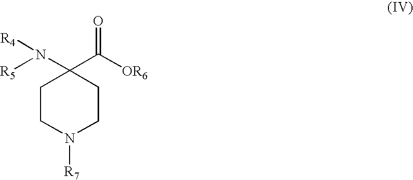
wherein R₇ is a hydrocarbyl or substituted hydrocarbyl; reacting the intermediate compound (IV) with an acylating agent to form a compound (V) having the formula: 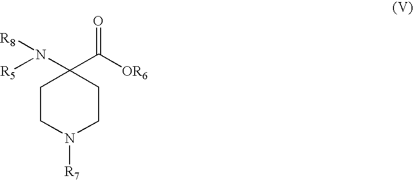
wherein R₈ is —C(O)R₉ and R₉ is hydrocarbyl or substituted hydrocarbyl.

38. A process for synthesizing an intermediate, the process comprising:
reacting compound (I) having the formula: 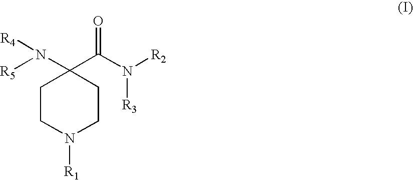
with a base in the presence of a solvent to form intermediate compound (II) having the formula: 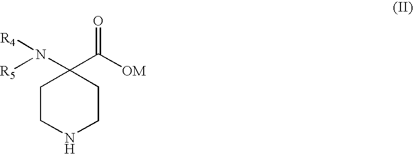
wherein R₁, R₂, R₃, R₄, and R₅ are independently selected from the group consisting of hydrogen, hydrocarbyl, and substituted hydrocarbyl and M is hydrogen or a cation.

39. A process for synthesizing an intermediate of an opiate or opioid analgesic or anesthetic, comprising:
reacting an intermediate compound (II) having the formula: 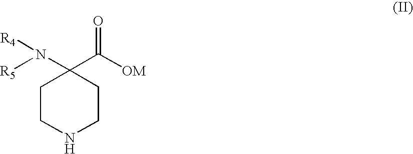
wherein R₄, and R₅ are independently selected from the group consisting of hydrogen, hydrocarbyl, and substituted hydrocarbyl and M is hydrogen or a cation, with an alcohol, R₆OH to form an intermediate compound (III): 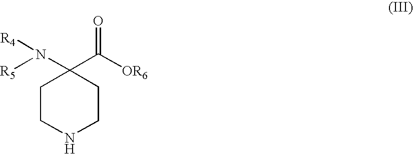
wherein R₆ is selected from the group consisting of hydrocarbyl and substituted hydrocarbyl.

40. The process of claim 37, wherein
R₁ is H, R₁₀O(O)C—, R₁₁O(O)CR₁₂—, R₁₁R₁₀—, R₁₀O(O)CR₁₂—, R₁₁R₁₀O(O)CR₁₂—, R₁₁(O)COR₁₂—, R₁₀(O)COR₁₂—, R₁₁R₁₀(O)COR₁₂—, R₁₀(O)CR₁₃OR₁₂—, or R₁₁(O)CR₁₃OR₁₂—;
R₂, R₃, R₄, and R₅ are independently H, cycloalkyl, substituted cycloalkyl, heterocyclic, R₁₄OR₁₅— or R₁₆R₁₅—;
R₁₀, R₁₂, and R₁₃ are independently hydrocarbyl or substituted hydrocarbyl;
R₁₁ is heterocyclic;
R₁₄ and R₁₅ are independently hydrocarbyl or substituted hydrocarbyl; and
R₁₆ is cycloalkyl, substituted cycloalkyl, or heterocyclic.

41. The process of claim 40, wherein R₁ is H, R₁₀(O)COR₁₂— or R₁₀O(O)C—; R₂ is an aryl with or without substitution; R₃ is H or lower alkyl; R₄ is H; R₅ is phenyl; and R₁₀ and R₁₂ are alkyl.

42. The process of claim 41, wherein R₁ is H or R₁₀(O)COR₁₂—; R₂ is phenyl; R₃ is H or methyl; R₁₀ is ethyl; and R₁₂ is methyl.

43. The process of claim 41, wherein R₁ is R₁₀O(O)C—; R₂ is phenyl; R₃ is H or methyl; and R₁₀ is ethyl.

44. The process of claim 37, wherein M is a metal cation selected from the group consisting of sodium, potassium, or lithium.

45. The process of claim 37 further comprising reacting the intermediate compound (II) with an acid before reaction with the alcohol R₆OH to form the intermediate compound (III).

46. The process of claim 37, wherein the reaction time for the formation of compound (II) is about 4 to about 48 hours and said solvent is selected from the group consisting of water, acetonitrile, acetone, dichloromethane, chloroform, n,n-dimethylformamide, dimethylsulfoxide, ethylacetate, dichloroethane, triethylamine, benzene, toluene, xylene, methanol, ethanol, isopropanol, n-propanol, 1-butanol, tert-butanol, 4-methyl-isopropanol, 1,4-dioxane, tetrahydrofuran (THF), 1,1-oxybisethane, nitrobenzene, and mixtures thereof.

47. The process of claim 37, wherein compound (II) is formed at a temperature of from about 120° C. to about 200° C.

48. The process of claim 47, wherein compound (II) is formed in a closed reaction chamber and the pressure in said closed reaction chamber is from about 4 atm to about 12 atm.

49. The process of claim 37, wherein said base is selected from the group consisting of a metal hydroxide, a metal hydride, an amine, ammonium hydroxide, and a quaternary alkyl ammonium hydroxide.

50. The process of claim 37, wherein R₆ is R₁₇OR₁₈—, R₁₉R₁₈—, or R₂₀R₁₈—, R₁₇ and R₁₈ are independently hydrocarbyl or substituted hydrocarbyl, R₁₉ is aryl or substituted aryl, and R₂₀ is cycloalkyl, substituted cycloalkyl or heterocyclic.

51. The process of claim 39, further comprising reacting compound (III) with an alkylating agent to form an intermediate compound (IV): 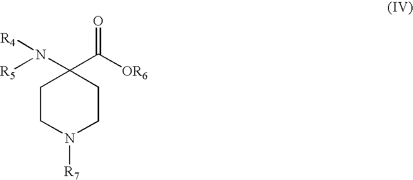
wherein R₇ is a hydrocarbyl or substituted hydrocarbyl.

52. The process of claim 51 further comprising reacting compound (IV) with an acylating agent to form a compound (V) having the formula: 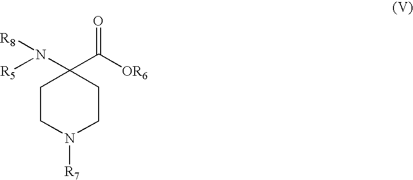
wherein R₈ is —C(O)R₉, and R₉ is hydrocarbyl or substituted hydrocarbyl.

53. The process of claim 37, wherein R₇ is selected from the group consisting of methyl propionate, ethyl propionate, 2-phenylethyl, 2-(2-thienyl)ethyl, and 2-(4-ethyl-4,5-dihydro-5-oxo-1H-tetrazol-1-yl)ethyl.

54. The process of claim 37, wherein R₇ is R₂₁OC(O)R₂₂—, R₂₁C(O)OR₂₂—, R₂₁OR₂₃OC(O)R₂₂—, R₂₄R₂₂—, or R₂₅R₂₂—; R₂₁, R₂₂, and R₂₃ are independently hydrocarbyl or substituted hydrocarbyl; R₂₄ is cycloalkyl or substituted cycloalkyl; and R₂₅ is heterocyclic.

55. The process of claim 37, wherein compound (III) is formed in the presence of a catalyst and/or desiccant.

56. The process of claim 37, wherein the alkylating agent is selected from the group consisting of methyl acrylate, ethyl acrylate, acrylic acid, acrylonitrile, acrylamide, acrolein, phenylethyl halide, tolylate, mesylate, styrene, and substituted styrene.

57. The process of claim 37, wherein the acylating agent is selected from the group consisting of ethanoyl chloride, propionyl chloride, propionic anhydride, methyl ketene, butanoyl chloride, and an alkyl acid cyanide.

58. The process of claim 37, wherein the acylating agent is propionyl chloride or propionic anhydride and compound (V) is remifentanil or carfentanil.

59. The claim 37, wherein the process is free of cyanide compounds.
